(19) United States
(12) Patent Application Publication
(10) Pub. No.: US 2026/0212968 A1
(43) Pub. Date: Jul. 23, 2026

(54) Data processing architecture

(71) Applicant: Flok Health Ltd, Cambridge (GB)

(72) Inventors: Finn Cameron James Stevenson, Cambridge (GB); Ricardo Michael Henderson Da Silva, Cambridge (GB)

(21) Appl. No.: 19/142,593

(22) PCT Filed: Dec. 22, 2023
(86) PCT No.: PCT/GB2023/053361

§ 371 Date: Jun. 23, 2025

(30) Foreign Application Priority Data
Dec. 23, 2022 (GB) 2219757.8

(51) Int. Cl. G16H 10/20; G16H 10/60; G16H 50/20; G16H 50/30; G16H 80/00

(52) U.S. Cl. CPC G16H 10/20; G16H 10/60; G16H 50/20; G16H 50/30; G16H 80/00

(57) ABSTRACT

A method for providing content to a patient, comprising: storing in memory accessible by one or more processors: (a) data defining a plurality of automated interventions and (b) a dataset representing mappings between (i) a patient state defined with respect to multiple patient parameters and (ii) one of the interventions; forming a set of requests to a patient; receiving from the patient in response to the requests a set of patient information; executing program code on the processor(s) to perform the steps of: forming a current patient state in dependence on the patient information; comparing the current patient state to the patent states of the dataset and in dependence on that comparison selecting an intervention, and providing the selected intervention to the patient.

This invention relates to data processing architectures and data transmission architectures.

It is known to store video content and play it out or stream it over a data processing network such as the internet. It is also known to capture live video content and to stream that content over a network as it is being generated. Distributing content so that it can be viewed within a few seconds of having been generated is referred to as real-time distribution.

Online sports coaching is becoming popular. With online sports coaching a trainer presents a workout which is distributed in real time to viewers. The viewers can participate in the workout at locations remote from the trainer. For example, if many viewers send messages to the trainer to say that the find the workout too hard or too easy then the trainer can adapt the workout accordingly. This format can increase levels of participation. Some viewers feel a greater sense of engagement with a real-time presentation than with a pre-recorded presentation. However, because the trainer is making a single presentation, the presentation is the same for all the viewers. Since the viewers might have different levels of ability, the workout might not be optimal for all the viewers.

Healthcare professionals can deliver individualised content to patients, but this occupies a substantial amount of the healthcare professionals'time, and is expensive to scale.

It would be desirable to be able to provide a presentation that can engender a similar level of engagement to a live presentation, and that can be adapted to the requirements of different viewers. Such a presentation might be distributed simultaneously, or at different times, to many participants. It can be anticipated that this would require significant data processing capacity to generate varied content and significant data transmission capacity to distribute the content.

According to one aspect there is provided a method for providing content to a patient, comprising: storing in memory accessible by one or more processors: (a) data defining a plurality of automated interventions and (b) a dataset representing mappings between (i) a patient state defined with respect to multiple patient parameters and (ii) one of the interventions; forming a set of requests to a patient; receiving from the patient in response to the requests a set of patient information; executing program code on the processor(s) to perform the steps of: forming a current patient state in dependence on the patient information; comparing the current patient state to the patient states of the dataset and in dependence on that comparison selecting an intervention, and providing the selected intervention to the patient.

The method may comprise selecting the intervention mapped to a patient state that is the closest of the stored patient states to the current patient state, and providing the selected intervention to the patient.

The method may comprise defining a series of automated intervention pathways, each pathway comprising a plurality of interventional steps; and storing each pathway as a respective one of the automated interventions.

The method may comprise defining a series of automated intervention pathways, each pathway comprising a plurality of interventional steps; and storing each pathway as a respective one of the automated interventions.

The method may comprise defining a series of automated intervention pathways, each pathway comprising a plurality of interventional steps; storing each of the interventional steps as a respective one of the automated interventions; and repeatedly receiving from the patient in response to the requests a set of patient information, forming a current patient state in dependence on that patient information; comparing that current patient state to the patent states of the dataset and in dependence on that comparison selecting an intervention, and providing the selected intervention to the patient.

The method may comprise, subsequent to providing a selected intervention to the patient, receiving feedback from the patient and in dependence on that feedback modifying the dataset.

Each automated intervention may comprise a messaging mechanism and therapeutic messaging.

The method may comprise modifying the dataset by modifying a mapping between (i) an intervention other than the selected intervention that has a messaging mechanism in common with the selected intervention and (ii) a patient state defined with respect to multiple patient parameters.

The method may comprise providing the intervention as a video.

According to a second aspect there is provided a system for automatically determining content to be presented to users, the system comprising one or more processors, the processors having access to (i) data identifying a set comprising multiple instances of presentational content and (ii) a data set storing for each of the instances of content data on each of multiple dimensions applicability information indicating the applicability of the respective content data to the respective dimension and the processors being configured to: receive a request for content from a user; receive data indicative of the user's state on each of the dimensions; compare the user's state to the applicability information for each of the instances of content and thereby determine an instance of content applicable to that user; cause the determined instance of content to be provided to the user for presentation; and receive feedback data indicative of the user's state subsequent to the said causing step; and adapt the applicability information for the determined content in dependence on the feedback data.

According to a third aspect there is provided a data carrier storing instructions for causing one or more processors to implement a system for automatically determining content to be presented to users, the system comprising one or more processors, the processors having access to (i) data identifying a set comprising multiple instances of presentational content and (ii) a data set storing for each of the instances of content data on each of multiple dimensions applicability information indicating the applicability of the respective content data to the respective dimension and the processors being configured to: receive a request for content from a user; receive data indicative of the user's state on each of the dimensions; compare the user's state to the applicability information for each of the instances of content and thereby determine an instance of content applicable to that user; cause the determined instance of content to be provided to the user for presentation; and receive feedback data indicative of the user's state subsequent to the said causing step; and adapt the applicability information for the determined content in dependence on the feedback data.

Each instance of content comprises interventional content. It may, for example, comprise instructions for a consumer of (e.g. viewer of or listener to) the content to perform an action. That action may be a physical or mental action. The action may be a physical exercise. It may be a therapeutic action. It may be a physiotherapeutic action. It may be a stretching and/or strengthening action.

The feedback data may comprise one or more of: whether the determined instance of content has been presented to the user, how much of the determined instance of content has been presented to the user, input provided by the user, data regarding the user's physical state as gathered by a computer vision system.

The system may, subsequent to the adapting step, receive a request for content from a user and determine an instance of content for presentation to a user in dependence on the adapted applicability information.

Each instance of content may be one of: video content, text, a template comprising time slots into which video content can be inserted. It may be a composite item of content comprising items of video assembled in accordance with such a template.

The processor(s) may be configured to, on determination of an instance of content that is text, generate a video dependent on that text and cause that video to be provided to the user for presentation.

The content presented to a user may be physiotherapy content.

The user state may comprise a musculoskeletal health condition.

The present invention will now be described by way of example with reference to the accompanying drawings.

In the drawings:

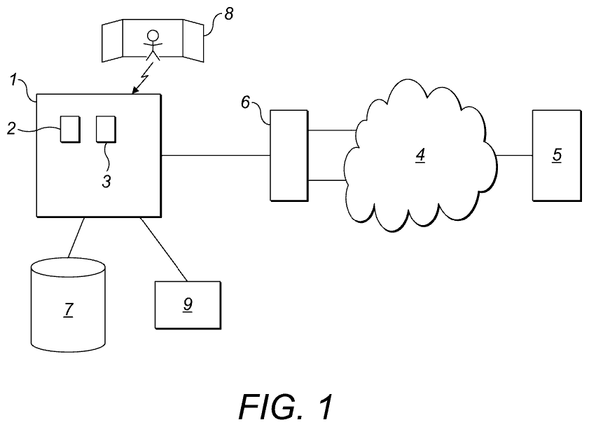

FIG. 1 shows first data processing architecture.

The architecture to be described below is suitable for generating video content that can be distributed to multiple viewers. The content can be customised so that some viewers receive different content to others. Potentially, each viewer may receive content that is different from that distributed to any of the other viewers.

Some examples will be given of how the logic by which the content can be formed. These examples are illustrative. Other ways of forming the content may be adopted.

The content can be generated by combining one or more of the following elements: (a) live video elements, (b) pre-recorded video elements and (c) computer-generated video elements. Live video elements are generated by human actors contemporaneously with the generation of the combined video content. Pre-recorded video elements are generated or captured prior to the generation of the combined video content and are stored so that they can be used to generate the combined content. Computer-generated video elements are generated by computer under the command of suitable software. They may be generated contemporaneously with generation of the combined video content. Computer-generated elements generated before the combined video content is generated may be stored as pre-recorded video elements.

To customise the content, elements identified above can be selected, ordered and/or positioned in a video frame depending on data specific to an intended viewer. The content can be customised in dependence on one or both of (a) viewer state information and (b) viewer feedback. The viewer state information is information that relates to the state of the viewer and has been captured before the video content is generated. It may, for example, include preferences of the viewer and/or data indicative of the viewer's previous levels of performance. The viewer feedback is data received from equipment associated with the viewer during the generation of the content. The viewer feedback may be entered into the equipment under the command of the viewer, for example by the viewer answering a question or writing a message. Alternatively the viewer feedback may be automatically captured in dependence on the viewer's behaviour, for example from a physiological sensor such as a heart rate monitor or from a camera capturing images of the viewer. The viewer feedback is transmitted from the equipment associated with the viewer to equipment that generates the video content.

The purpose of the content may be to instruct or advise a viewer on an activity. That may be a physical activity and/or a mental activity. The nature of the instruction may be adapted to the viewer in dependence on viewer state information for that viewer and viewer feedback received from that viewer. In one example, the purpose of the content may be to provide therapeutic instruction to the viewer. The instruction may be to perform certain stretches or strength exercises for physiotherapeutic purposes. In another example, the instruction may be to perform certain mental tasks such as relaxation or cognitive challenges. For illustration, if the purpose of the content is to provide therapeutic instruction then the elements from which the content can be formed may be as follows:
(a) Live video elements comprising video of a human presenter. The presenter may introduce other components of the content.
(b) Pre-recorded video elements comprising video depicting a human performing physical stretches or exercises. Pre-recorded video elements may also comprise video comprising computer-generated imagery depicting the performance of physical stretches or exercises.
(c) Computer-generated video elements which may, for example, be contemporaneously generated imagery depicting the performance of physical stretches or exercises or describing such stretches or exercises.

Apparatus for generating and distributing the content will now be described. The apparatus may form the content at one or more locations. From those locations the content can be distributed to viewers, for example by being streamed over a network such as the internet. The apparatus may be remote from the viewers. Each viewer may use a viewing device to view the content. That viewing device may, for example, be a phone, tablet, computer or television. The viewing device comprises one or more processors and memory storing code executable by the processor(s) to play back the content. The memory stores the code in a non-transient manner. The viewing device may implement a dedicated mobile application or app or a web browser for playing back the content.

As indicated above, the content may be played back in real time, for example live elements of the content may be played back within 10 seconds or 5 seconds of having been generated and/or within 10 seconds or 5 seconds of elements of the content having been combined. This can allow a viewer to provide feedback on the content that can have a substantially immediate effect on the generation of the content. That can enhance a feeling of engagement on the part of the viewer. Alternatively, or in addition, the content can be generated and then stored by the generating apparatus and then distributed and played out later to a viewer.

Some non-limiting examples of the type of content that may be provided by the present system will now be discussed. The content may provide directions and/or information for therapeutic, fitness, wellbeing or rehabilitation purposes. It may demonstrate exercises, routines or poses. The content may be directed to the treatment of one or more medical conditions. Subjects suffering from a common condition, for example back pain, may vary greatly in their capacity to undertake certain stretches, poses or strength exercises. For that reason, if a set of such subjects are provided with the same instructions for performing a series of those movements it is possible that the set of movements will be unsuitable for a proportion of the subjects. For some of the subjects the movements might be unachievable. For others of the subjects the movements might be too easy to bring about a substantial improvement in symptoms. There may be a risk that some subjects may suffer injury from undertaking unsuitable movements. This is a situation in which it may be advantageous to provide customised video content as described herein. By providing video content in this way, reliance on individual healthcare professionals to provide one-to-one therapy can be reduced, and it may be possible to deliver individualised treatment more economically to a greater number of people.

In such a system, advice may be automatically delivered to a patient by the following steps.

In the manner to be described below, a server provides content and other messaging and interaction to the patient over a suitable network, for example the internet. The patient interacts with the server using a client device such as a phone, tablet or laptop, optionally via a dedicated application running thereon. In an initial step the patient establishes an account on the system and selects from available options and/or answers questions posed by the system. The patient may be invited to draw on a screen where they experience pain. These interactions provide information by which the system can assess the patient's condition and its severity. Using that information the system can select an initial set of custom content for delivery to the patient.

The content is suitably delivered to the patient in the manner of an online class. This involves a stream depicting a live or real-time presenter, or a stream of a presenter who has been pre-recorded but appears to be live. In the manner described herein, such a stream of the presenter is combined with other elements. In practice this may involve the presenter providing an introduction that is generic to multiple exercises, which the system then combines with a pre-recorded and/or CGI element depicting an exercise. The system selects the exercise element based on information about the state of the patient and/or feedback received from the patient during the playout of the content. In this way, the patient can receive content that depicts the presenter in an ostensibly live setting, together with exercise or other content that is selected for the patient in a custom fashion. This can improve the patient's engagement with the content whilst providing a set of exercises or other recommendations that are suitable for the patient's ability. In one example, the exercise element may be overlain on a portion of the video frame whilst at least some of the remainder of the frame depicts the presenter. In another example, the custom content may cut from the presenter to the exercise element.

Where a CGI element is used, it may be generated in any of a number of ways. One option is for the CGI element to be generated in advance of playout and stored as a video clip. Another option is for the CGI element to be stored as a definition for input to a CGI algorithm: for example as a set of joint motions for a human model. Then that definition can be implemented during playout to form the CGI contemporaneously. Another option is to store a natural language or narrative prompt that describes the desired content of the CGI element. When it is desired to generate and/or provide the CGI content the prompt can be fed to a generative video system which is configured to generate video from such prompts. Examples of such systems include GAN-based neural networks. Such a system can the output the desired video content for delivery to one or more participants. One advantage of this system is that the prompts may require less storage space than pre-generated video content. Another advantage may be that the stored prompt can be augmented, at the time of processing by the generative video system, with other information that can affect the generated video, for example to make it more relevant to the intended participant. For example, the prompt may be augmented with cultural, gender, age or appearance information known about the participant, and the generative video system can generate the video accordingly.

With suitable phrasing of the presenter's script, the presenter's presentation may blend with a range of exercise elements. For example, the presenter may say: “let's start with this stretch”, and then the system may incorporate an exercise element selected to be suitable for the individual patient or viewer to whom a specific set of combined content is to be served. At that point each patient may see an element depicting a different stretch, selected to be suitable for the level of that patient. In that way, multiple patients can be engaged by a common live or ostensibly live presenter whilst receiving customised content that is convincingly combined with the presenter's comments. In another example, the presenter may say “now try this exercise, which helps to improve your mobility”. Then each patient may see an exercise element that is selected specifically for them; and whilst all the exercise elements that the system inserts at this point are for improving mobility, they may be at different levels of difficulty for each patient.

To achieve this automatically, first the available exercise elements may be classified into different types, for example stretches, back lateral mobility exercises and lower back strength exercises. They may also be assigned a difficulty, or the system may learn the difficulties of each one over time through feedback from patients. Then a healthcare professional designs a therapy session by selecting a series of exercise element types which will be presented in turn. Timings may be assigned to each exercise element so that they can be synchronised with the presenter's presentation. Data defining the session is input to the system to permit it to select the exercise types during the generation of custom content. It is also provided to the presenter who can use it to form a script. Then during the generation of the content the presenter can present that script and the system can select and incorporate at the designated times suitable exercise elements for each patient. During the playout of the content each patient may provide feedback, for example on how hard they found each exercise, or on the level of their symptoms. This system can use this information for multiple purposes, for example (a) to refine its record of the difficulty or perceived difficulty of each exercise, for example through questions such as “how hard was this for you today”, and (b) to assess whether over a cohort of patients a certain exercise or exercise type tends to provide a therapeutic improvement. This information can be used to improve future content generation.

In the presentation or through other communication channels messages may be given to a patient to encourage their continued engagement with the system, for example through tailoring wording for each patient, automatically determining what notifications to deliver to a patient and when, motivational interviewing such as periodically asking or reminding patients about quality-of-life elements that they are working towards, gamification of treatment attendance e.g. through streaks, leaderboards, social accountability, and by automatically determining when to provide a patient with additional interventions: for example by a healthcare professional contacting a patient directly if it is determined that the patient is demotivated or losing engagement.

FIG. 1 shows an architecture for generating content. The architecture comprises a server 1. The server comprises one or more processors 2 and memory 3 storing in non-transient form code executable by the processor(s) to perform the functions described herein. The server may be located in a single place or it may be distributed between multiple physical units. The server can provide content over a network 4, such as the internet, to consuming devices 5. The consuming devices may be mobile phones, tablets, televisions, personal computers or any other suitable devices. To assist in serving content to large numbers of consuming devices, the server may connect to the network 4 via a fanout server 6. The fanout server can reproduce a content stream received from the server 1 and provide multiple streams to the network 4. Content to be played out by the server can be stored or generated in multiple ways. For example, the server 1 can receive content stored in a database 7; it can receive live content from a studio 8; or it can receive computer-generated content from another server 9, for example a generative video server. As will be described below, the processor(s) of the server receive requests for content from intending consumers. In response to those requests, and to (a) data stored that provides information about the person requesting the content and (b) pre-defined content and/or content templates, the server can generate content to be served to the consumers. That content may be individualised to the consumers in ways to be described below.

A suitable architecture may be scalable so that individualised content can be delivered to large numbers of viewers, for example thousands or hundreds of thousands of viewers. The server 1 may implement a core application. This provides interfaces to users who can, among other actions, configure the logic to be used by the system to form the content. The core application can control the storage of elements to be included in the content. The core application can combine the elements to form content and play that content out to viewers. The core application can receive and process viewer feedback. The core application can combine video elements in dependence on the viewer feedback and/or on other data stored for a respective viewer. Hardware at studio 8 can be used to capture video of a presenter or a person performing an exercise. The combined content can conveniently be played out to viewers over a content delivery network (CDN) 4, 6. Viewers may use a local device 5 running suitable software to play out content that is streamed to them and to capture feedback and transmit that feedback to the core application on server 1.

A relational data structure may be used for storing data accessible by the core application. A cohort of viewers and potential viewers includes multiple patient records. For each patient records data sets are stored indicating the user's preferences, prior interactions with the system, prior levels of performance and the like. The system may collect longitudinal or time-series data regarding interactions of a viewer/patient with the system.

Core application services may be provided on a cloud computing platform. This together with other features can help to improve the scalability of the system.

Those other features may include:
The provision of mixer servers to combine a live input stream element with pre-recorded video elements.
Storing pre-recorded video elements on suitably fast drives or on memory on mixer servers.
The provision of a fanout server to receive the upload and distribute to mixer servers.
Combining the video elements on serverless edge compute platforms. This may assist in allowing them to be scalable without the burden of infrastructure management.

A content distribution system may assist in ensuring that each viewer receives the correct stream.

The system may implement an API to provide viewer devices with authenticated unified resource identifiers (URIs) for each item of video content. This can enhance the security of the system.

The video may be transmitted in any suitable format. Conveniently the format may be selected so as to reduce latency without substantially compromising quality. Examples of suitable formats may include HLS, e.g. LL-HLS, and WebRTC. The SRT format may be used to upload pre-recorded video elements.

The core application can form the combined content by any suitable technique. Examples include (a) forming a first part of the combined content from one video element and forming a second part of the combined content occurring after the first part from another video element and (b) forming a part of the combined content by overlaying one video element on another. The former technique may employ frame generation to provide smooth transitions between elements. Examples may include IFRNet and FeatureFlow. The latter technique may employ known video processing algorithms for background or scene replacement. To enhance the quality of the combined content, the core application may process one or more elements to adjust their brightness, contrast, white balance, audio volume and other factors so that the elements appear more harmonious when played out.

As indicated above, viewers may interact with the system while watching the playout of content. Viewer's interactions may be received and processed in several ways. Examples of such ways include:
Speech-to-text—prompting the viewer to respond verbally to questions or prompts from the live or pre-recorded presenter. The received audio may be processed on the viewer's device or by a cloud computing application.
Question and/or response buttons displayed on the viewer's device
Data collected from a wearable device such as a smart watch
Data collected from a camera imaging the viewer, for instance a camera on the viewer's device

To increase engagement with the video content, gamification techniques may be used. These may include:
recording and displaying information indicative of the viewer's previous performance;
recording and displaying information indicative of the viewer's current performance in comparison to the current or prior performance of the same or other viewers;
recording a viewer's history of interaction with the system and displaying data indicative thereof;
displaying a statistical representation of other viewers currently receiving the content or related content.

In an example architecture, a first cloud entity (VPC) includes one or more elastic cloud service (ECS) entities. Each ECS entity provides a mixer service and a fanout service. The mixer processes viewer state data and viewer feedback data and in dependence on one or both of those selects and combines video elements as described above to form individualised content for one or more viewers. The fanout service receives a live video feed and distributes it to the mixer service. The outputs of the mixer service are streamed to viewers. This architecture may be advantageous in allowing the system to be readily scaled to accommodate larger numbers of users. First, if the ECS is provided by a cloud computing platform, it may be straightforward to provide more instances of the ECS to accommodate more viewers. Second, the fanout service can replicate the live stream to multiple feeds. This can allow the live stream to be combined more readily with other video elements. With a fanout arrangement, horizontal scalability can efficiently permit scaling of the total number of distributed streams.

In an example of a video processing subsystem, a live presentation is captured by a camera. The output of the camera is provided to a hardware encoder. The hardware encoder is hardware that transcodes the video feed from the camera into a suitable format. In one example, the output of the hardware encoder may consume 1.5 MB/s. The output of the hardware encoder is a video stream representing the live presentation. That output is passed to a fanout server. The fanout server replicates the video stream representing the live presentation to generate multiple parallel video streams representing the same live presentation. The fanout server may be a stand-alone hardware entity. The parallel streams are provided to mixer services. Each mixer service may form multiple content streams for viewers. Each content stream may comprise part or all of the live presentation combined with one or more other video elements of the types described above. Those other elements may be stored as pre-recorded chunks at or locally to the mixer service. Each mixer service may receive a respective one of the parallel video feeds. This architecture can help to allow the individual viewer feeds to be formed efficiently. It can also allow the system to be scaled readily. Additional mixer services can be provided to allow more viewer streams to be generated. Because the mixer service is a software entity, it is relatively easy to provide more mixer services as required. The system may have a management entity for increasing or decreasing the number of instances of the mixer service to meet viewer demand. The management entity may assess how many viewers are requesting video feeds, and dependant on that increase or decrease the number of mixer entities. The number of fanout servers, which may be provided by dedicated hardware, and their outputs may remain unchanged.

From time to time a user may engage with the system to request to consume content. For example the user may open a mobile application or log on to the system through a web browser and indicate a readiness to view video content. A processor of a server of the system can then select suitable content or a suitable template for content to be served to that user. The content may be selected in any convenient way. However, the following techniques may be advantageous.
1. The system may store one or more predefined pathways. Each pathway may comprise multiple items of content or content template. Based on information gathered from or about the user the user may be assigned to a pathway, and a starting position for the user on the pathway may be determined. Then the user is presented with the content for that position on that pathway. In subsequent interactions, that user may be presented with the content for successive positions on the pathway, or subsequent content may be determined as described below. As an example of this approach, the system may store five videos or content templates relating to back pain. The videos or templates may present exercises of increasing difficulty. Based on an initial assessment of a user it may be determined that the user is suffering from back pain. From that determination the user may be assigned to the back pain pathway. The user's back pain may be assessed as moderate. From that assessment the user may initially be shown the third video or template in the sequence. In subsequent interactions the user may be shown the fourth and fifth videos or templates. Progression may be dependent on the user being assessed to have improved.
2. The system may store (i) data indicative of the state of the user, (ii) a set of videos or templates, and (iii) data indicating the states of users who have been presented with each video or template in the past together with outcome data indicative of the outcome of each such presentation. For example, the outcome data may indicate one or more of the following factors: whether all of the video or template content was viewed by the user to whom it was to be presented, whether that user gave positive feedback after the presentation of the video or template content, and whether a condition experienced by that user improved after the presentation. Other factors may also be adopted. When a user requests to consume content, data on the state of that user ((i) above) may be compared with the states of users who have previously been presented with content (in (iii) above). A server of the system can use a predefined algorithm to select content that when presented to users of a similar state to the requesting user has been associated with a positive outcome (from the data in (iii) above). The system may balance the similarity of user state and the degree of positivity of outcome in a manner selected by an operator of the system so as to achieve a desired balance between relevance of content and expected positivity of outcome. The system selects the video or template that provides the best fit on the predetermined metric and presents that to the user. This approach allows the system to benefit from data gathered about outcomes and relevance of content for other users. In practice, with this approach a user need not remain on a given pathway if that pathway is no longer the apparently most beneficial for the user. For example, a user may previously have been presented with the third video on the back pain pathway. On requesting further content the system may determine that the state of the user is such that whilst their back pain remains moderate they now have severe shoulder pain. By balancing the assessment of state with the previous outcomes for users with comparable states to the requesting user the system may determine that the best video to be presented is now a video relating to shoulder pain. Alternatively, it may determine that the most positive effects for users in a comparable state has been associated with continued presentation of a video related to back pain. It can be imagined that this might be the case if there is a likelihood that the shoulder pain of a user with this state is related to an underlying condition affecting their back.

The server may be a single computing entity or it may be physically distributed over multiple locations. The server may comprise one or more processors. The processors may be configured to execute code stored in non-transient form to perform the actions set out herein.

There may be a data carrier, for example a memory, storing in non-transient form code executable by the processor(s) of the server to perform the actions set out herein.

In one approach, the initial content to be presented to a user can be determined by approach 1 above, and subsequent content can be determined by option 2. In another approach, approach 2 above can be used also for determining the initial content.

Approach 2 can have the advantage that the system can adapt automatically to changes in a user's state. The system can present content that has been found to be the most beneficial to users of a similar state to the requesting user. The requesting user need not stay on a predetermined pathway if that pathway is no longer the most relevant to the user. If the user does remain on a pathway, their progression on that pathway can be automatically assessed by approach 2 above.

In a first variant of approach 2, the system may store for each video or template data indicating the state of a user for whom that content is intended. Then the system may select the content whose stored state most closely matches the state of the requesting user. This can simplify the process of determining which content to present.

In a second variant of approach 2, the state data stored for an instance of content may be indicative of the applicability of that instance of content to a user of a given state. The state data may comprise data indicating, on each of multiple data dimensions, the applicability or suitability or relevance of that instance of content for a user having a state in that dimension. Instances of content appropriate for a user requesting content may then be identified by searching for the instance of content whose stored state most closely matches that of the user according to a predetermined algorithm. Then, once the instance of content has been transmitted to a user for presentation and outcome data has been received, the state data for that item of content may be adapted in accordance with the received outcome data, for example by strengthening the link indicated by the state data with the state of the user prior to the delivery of the content if the outcome data indicates a positive outcome. In this way the system can learn which content is most appropriate or effective for a user in a given state. Initially the state data can be set by an operator. This can help avoid the content being presented inappropriately and can help accelerate the learning process.

In instance of content may, for example be a video clip or a template for video and/or live clips, or a definition, or example in the form of text, from which video representing the instance of content can be created, for instance using an automated text-to-video processing engine.

The state of the user, and of other users who have previously been presented with content, may be determined by the user answering questions posed by the system, by an automated assessment of the user derived from a photograph or video of the user or from an assessment by an expert, for example a healthcare professional.

The video or other content, which may be a part of a template as described above, may comprise an intervention. An intervention may be data that can alter the state of a person consuming the data. For example, it may be informational or instructional. It may comprise instructions to perform an activity, which may be a physical or mental activity. It may comprise instructions to perform the activity contemporaneously with the presentation of the content or subsequently.

In a system using approach 2 or a variant thereof, as described above, rather than a patient being presented with content from a predetermined pathway, self-contained items of content can be defined in advance. Then each time a user interacts with the system to consume content the system may select a preferred item of content to present to that user in dependence on the user's state at the time of the interaction. This means that a user can still be presented with appropriate content if (a) the user's progress is faster or slower than normal and/or (b) the user's principal requirement changes (e.g. if they develop a new condition that is more serious than an original condition).

A system as described above may be used for delivering content of any suitable media format. A system as described above may be used for delivering content of any suitable media content. For example, the media may be in video and/or audio form. The media may depict instructional content, for example instructions to perform therapeutic activities.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

The phrase “configured to” or “arranged to” followed by a term defining a condition or function is used herein to indicate that the object of the phrase is in a state in which it has that condition, or is able to perform that function, without that object being modified or further configured.

1. A method for providing content to a patient, comprising:
storing in memory accessible by one or more processors: (a) data defining a plurality of automated interventions and (b) a dataset representing mappings between (i) a patient state defined with respect to multiple patient parameters and (ii) one of the interventions;
forming a set of requests to a patient;
receiving from the patient in response to the requests a set of patient information;
executing program code on the processor(s) to perform the steps of: forming a current patient state in dependence on the patient information; comparing the current patient state to the patent states of the dataset and in dependence on that comparison selecting an intervention, and providing the selected intervention to the patient.

2. A method as claimed in claim 1, comprising selecting the intervention mapped to a patient state that is the closest of the stored patient states to the current patient state, and providing the selected intervention to the patient.

3. A method as claimed in claim 1 or 2, comprising:
defining a series of automated intervention pathways, each pathway comprising a plurality of interventional steps; and
storing each pathway as a respective one of the automated interventions.

4. A method as claimed in claim 1 or 2, comprising:
defining a series of automated intervention pathways, each pathway comprising a plurality of interventional steps;
storing each of the interventional steps as a respective one of the automated interventions; and
repeatedly receiving from the patient in response to the requests a set of patient information, forming a current patient state in dependence on that patient information; comparing that current patient state to the patent states of the dataset and in dependence on that comparison selecting an intervention, and providing the selected intervention to the patient.

5. A method as claimed in any preceding claim, comprising, subsequent to providing a selected intervention to the patient, receiving feedback from the patient and in dependence on that feedback modifying the dataset.

6. A method as claimed in any preceding claim, wherein each automated intervention comprises a messaging mechanism and therapeutic messaging.

7. A method as claimed in claim 6 as dependent on claim 5, comprising modifying the dataset by modifying a mapping between (i) an intervention other than the selected intervention that has a messaging mechanism in common with the selected intervention and (ii) a patient state defined with respect to multiple patient parameters.

8. A method as claimed in any preceding claim, comprising providing the intervention as a video.

9. A method as claimed in any preceding claim, wherein the content presented to a patient is physiotherapy.

10. A method as claimed in any preceding claim, wherein the patient state comprises a musculoskeletal health condition.

11. A system for automatically determining content to be presented to users, the system comprising one or more processors, the processors having access to (i) data identifying a set comprising multiple instances of presentational content and (ii) a data set storing for each of the instances of content data on each of multiple dimensions applicability information indicating the applicability of the respective content data to the respective dimension and the processors being configured to:
receive a request for content from a user;
receive data indicative of the user's state on each of the dimensions;
compare the user's state to the applicability information for each of the instances of content and thereby determine an instance of content applicable to that user;
cause the determined instance of content to be provided to the user for presentation; and
receive feedback data indicative of the user's state subsequent to the said causing step; and
adapt the applicability information for the determined content in dependence on the feedback data.

12. A system as claimed in claim 11, wherein each instance of content comprises interventional content.

13. A system as claimed in claim 11 or 12, wherein the feedback data comprises one or more of: whether the determined instance of content has been presented to the user, how much of the determined instance of content has been presented to the user, input provided by the user, data regarding the user's physical state as gathered by a computer vision system.

14. A system as claimed in any of claims 11 to 13, comprising, subsequent to the adapting step, receiving a request for content from a user and determining an instance of content for presentation to a user in dependence on the adapted applicability information.

15. A system as claimed in any of claims 11 to 14, wherein each instance of content is one of: video content, text, a template comprising time slots into which video content can be inserted.

16. A system as claimed in claim 15, wherein the processor(s) are configured to, on determination of an instance of content that is text, generate a video dependent on that text and cause that video to be provided to the user for presentation.

17. A system as claimed in any of claims 11 to 16, where the content presented to a user is physiotherapy content.

18. A system as claimed in any of claims 11 to 17, wherein the user state comprises a musculoskeletal health condition.
